(19) United States
(12) Patent Application Publication
(10) Pub. No.: US 2017/0249821 A1
(43) Pub. Date: Aug. 31, 2017

(54) FALL RISK ASSESSMENT DEVICE AND METHOD

(71) Applicant: Orthocare Innovations LLC, Edmonds, WA (US)

(72) Inventors: Kim Lisa Coleman Boone, Seattle, WA (US); David Alan Boone, Seattle, WA (US); Adam Arabian, Seattle, WA (US); Michael Orendurff, Kirkland, WA (US)

(73) Assignee: Orthocare Innovations LLC, Edmonds, WA (US)

(21) Appl. No.: 15/284,147

(22) Filed: Oct. 3, 2016

Related U.S. Application Data
(63) Continuation of application No. 13/276,949, filed on Oct. 19, 2011, now abandoned.
(60) Provisional application No. 61/394,648, filed on Oct. 19, 2010.

(51) Int. Cl. G08B 21/04; G06F 19/00; A61B 5/11; G01C 22/00; A61B 5/00

(52) U.S. Cl. CPC G08B 21/043; G08B 21/0446; G01C 22/006; A61B 5/7275; A61B 5/112; A61B 5/746; G06F 19/3431; A61B 2562/0219; G06F 19/3481; G06F 19/3418

(57) ABSTRACT

A method for assessing the risk of a patient to fall. The method includes attaching a pedometer on a patient, wherein the pedometer includes one or more sensors, allowing the patient to engage in activities throughout a predetermined period of time in, at least, an environment the patient occupies for a majority of the day while the pedometer senses information relating to steps taken by the patient. With one or more computers or with the pedometer, calculating at least one step variable from the acceleration information. With one or more computers or with the pedometer, comparing the at least one calculated step variable to a model step variable, and with one or more computers. Then, providing an assessment of the risk of the patient to fall. The pedometer may alert the patient when a risk of falling is detected.

### CROSS-REFERENCES TO RELATED APPLICATIONS

This application is a continuation of U.S. patent application Ser. No. 13/276,949, filed Oct. 19, 2011, which claims the benefit of U.S. Provisional Application No. 61/394,648, filed Oct. 19, 2010, both of which applications are expressly incorporated herein by reference.

### STATEMENT OF GOVERNMENT LICENSE RIGHTS

This invention was made with Government support under Grant No. R43HD056613 awarded by the Department of Health and Human Services National Institutes of Health. The Government has certain rights in the invention.

### BACKGROUND

Falls of elderly persons are often associated with severe declines in individual health and quality of life. Falling is an enormous public health problem with billions in direct costs attributable to such falls. However, an assessment of the risk of falling is inherently complex because of the myriad intrinsic and extrinsic factors that may contribute to the probability of a fall in any individual. Typically, a fall risk evaluation is performed by a clinician at a clinic or office. The clinician is trained to observe certain abnormalities or signs of imbalance when a patient walks a predetermined course. The clinical evaluation may be biased because the patient is in the artificial environment. Furthermore, the evaluation is inherently subjective owing to the experience of the clinician and the interpretation of abnormalities. Furthermore, no single source of information can provide the complete analysis of the risk of falling for an individual.

Accordingly, there is a long felt need for a device and method that can objectively assess the risk of falling in persons before, and regardless of whether, they have actually fallen yet.

### SUMMARY

In one embodiment, using the pedometer disclosed herein, the effects of extrinsic factors, such as the home environment, will be reflected by the device capturing the interaction between the subject and the environment, not physiological problems. Environmental challenges such as uneven surfaces or dim lighting have shown significant alterations to gait performance in persons prone to falling even when the same persons could not be differentiated when walking in a well-lit environment with a smooth floor. By monitoring gait when the patient is in his or her community and home environment, the pedometer will be measuring gait performance under the actual conditions that put the elder patient at risk to falling. The pedometer disclosed herein is an additional or alternative tool to identify those elder patients for whom more comprehensive and specific fall prevention action may be warranted, including environmental remediation to reduce gait challenges, which is itself a primary fall prevention intervention.

In one environment, the pedometer can be worn on the leg, such as the ankle, and used for multiple days during normal activities of daily life, producing a unique data set that better represents the fall risk of the subject in their normal activities rather than in the structured office environment. This long-term data collected is then downloaded by the treating user/clinician for review and analyzed automatically for fall risk probability.

In one embodiment, a method for assessing the risk of a patient to fall is disclosed. The method includes attaching a pedometer on a patient, wherein the pedometer includes one or more sensors, allowing the patient to engage in activities throughout a predetermined period of time in, at least, an environment the patient occupies for a majority of the day while the pedometer senses information relating to steps taken by the patient, with one or more computers calculating at least one step variable from the acceleration information, with one or more computers comparing the at least one calculated step variable to a model step variable, and with one or more computers providing an assessment of the risk of the patient to fall.

The model step variable may be compiled from information of persons considered to be samples of low risk of falling.

The information may include information of acceleration along one or more orthogonal axes of the patient's foot.

The information may include information of rate of turn of the patient.

The method may further include calculating a variability of stride duration of the patient and comparing to a variability of a model stride duration compiled from a group of persons characterized at low risk of falling.

The method may further include calculating a variability of stance phase duration of the patient and comparing to a variability of a model stance phase duration compiled from a group of persons characterized at low risk of falling.

The method may further include calculating a variability in accelerations in three orthogonal axes of the patient and comparing to a variability of model accelerations in three orthogonal axes compiled from a group of persons characterized at low risk of falling.

The method may further include calculating a variability in stride length of the patient and comparing to a variability of a model stride length compiled from a group of persons characterized at low risk of falling.

The method may further include calculating a rate of turn variable and comparing to a rate of turn variable compiled from a group of persons characterized at low risk of falling.

The method may further include detecting a stumble.

The method may further include transferring recorded information from the pedometer to one or more computers and, with the one or more computers, calculating the at least one step variable from the information.

The method may further include transferring recorded information from the pedometer to the one or more computers and, with the one or more computers, comparing the at least one step variable to the model step variable.

The method may further include two or more computers that are connected to a network and transferring the assessment of the risk of the patient to fall over the network from a first computer to a second computer.

In one embodiment, a method for alerting a patient of a risk of falling is disclosed. The method may include attaching a pedometer on a patient, wherein the pedometer includes one or more sensors and a processor, allowing the patient to engage in activities in, at least, an environment the patient occupies for a majority of the day while the pedometer senses information relating to steps taken by the patient, with the pedometer, calculating at least one step variable from the information, with the pedometer, comparing the at least one calculated step variable to a model step variable compiled from a group of persons characterized at low risk of falling, and, when a risk of falling is detected, the pedometer alerts the patient.

The method may further include providing an auditory or visual alert.

The information may include information of acceleration along one or more orthogonal axes, such as three.

The information may include information of rate of turn of the patient.

The method may further include calculating a variability of stride duration of the patient and comparing to a variability of a model stride duration compiled from a group of persons characterized at low risk of falling.

The method may further include calculating a variability of stance phase duration of the patient and comparing to a variability of a model stance phase duration compiled from a group of persons characterized at low risk of falling.

The method may further include calculating a variability in accelerations in three orthogonal axes of the patient and comparing to a variability of model accelerations in three orthogonal axes compiled from a group of persons characterized at low risk of falling.

The method may further include calculating a variability in stride length of the patient and comparing to a variability of a model stride length compiled from a group of persons characterized at low risk of falling.

The method may further include calculating a rate of turn variable and comparing to a rate of turn variable compiled from a group of persons characterized at low risk of falling.

The method may further include detecting a stumble.

In one embodiment, a pedometer is disclosed. The pedometer may include a housing, one or more sensors and a processor within the housing, a storage unit within the housing, the storage unit comprising a tangible computer readable medium having stored thereon instructions for calculating at least one step variable from information relating to steps, comparing the at least one calculated step variable to a model step variable compiled from a group of persons at low risk of falling, detecting a risk of falling, and, when a risk of falling is detected, alerting the patient.

The one or more sensors may include a triaxial accelerometer that measures acceleration along three orthogonal axes.

The one or more sensors may include a rate gyro that measures the rate of turn.

The tangible computer readable medium may further include instructions for calculating a variability of stride duration of the patient and comparing to a variability of a model stride duration compiled from a group of persons characterized at low risk of falling.

The tangible computer readable may further include instructions for calculating a variability of stance phase duration of the patient and comparing to a variability of a model stance phase duration compiled from a group of persons characterized at low risk of falling.

The tangible computer readable medium may further include instructions for calculating a variability in accelerations in three orthogonal axes of the patient and comparing to a variability of model accelerations in three orthogonal axes compiled from a group of persons characterized at low risk of falling.

The tangible computer readable medium may further include instructions for calculating a variability in stride length of the patient and comparing to a variability of a model stride length compiled from a group of persons characterized at low risk of falling.

The tangible computer readable medium may further include instructions for calculating a rate of turn variable and comparing to a rate of turn variable compiled from a group of persons characterized at low risk of falling.

The tangible computer readable medium may further include instructions for detecting a stumble.

In one embodiment, a method for assessing the risk of a patient to fall is disclosed. The method includes, attaching a pedometer on a patient, wherein the pedometer includes one or more sensors, allowing the patient to engage in activities throughout two or more predetermined periods of time in, at least, an environment the patient occupies for a majority of the day while the pedometer senses information relating to steps taken by the patient, with one or more computers calculating at least one step variable from information sensed during a first period of time, with one or more computers comparing the at least one calculated step variable to a step variable calculated from step information of the patient from a second period of time, and with one or more computers providing an assessment of the risk of the patient to fall. The one or more computers can be incorporated into the pedometer.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagrammatical illustration of a system for assessing the risk of falling;

FIG. 2 is a schematic illustration of a pedometer;

FIG. 3 is a schematic illustration of a computer;

FIG. 4 is a schematic illustration of a computer;

FIG. 5 is a flow diagram of a method for assessing the risk of falling;

FIG. 6 is a flow diagram of a method for assessing the risk of falling;

FIG. 7A is a representative graph of anterior/posterior acceleration for a stride;

FIG. 7B is a representative graph of medial/lateral acceleration for a stride;

FIG. 7C is a representative graph of axial acceleration for a stride;

FIG. 8A is a representative graph of the mean (center line) of anterior/posterior acceleration for a stride with the standard deviation upper and lower spread;

FIG. 8B is a representative graph of the mean (center line) of medial/lateral acceleration for a stride with the standard deviation upper and lower spread;

FIG. 8C is a representative graph of the mean (center line) of axial acceleration for a stride with the standard deviation upper and lower spread;

FIG. 9 is a diagrammatical illustration of one embodiment of a pedometer; and

FIG. 10 is a diagrammatical illustration of an exploded view of the pedometer of FIG. 9.

### DETAILED DESCRIPTION

Disclosed herein are a system and a method for assessing the risk of falling of a person by measuring step variables of a person's gait. Gait is the manner in which a person walks. Step variables describe numerical characteristics of a person's stride. A stride as used herein means one gait cycle, i.e., the two consecutive steps from the right and left foot. The system compares one or more variables to a model variable that is compiled from gait data of persons considered to be at low risk or have no risk of falling. The system and method allows longer term monitoring out of the office for detection of gait abnormality taking into consideration the challenges of everyday life. Duration induced fatigue effects may have important bearing on clinical interpretation of the muscle strength of elderly patients. Declining function is measured by comparison of multiple recordings over time that are stored on the computer media. Detection of declining functional ability with limitations in ambulation can be measured as variability and balance of step activity. This is accomplished by providing a pedometer with sensors, a storage unit, and a processor having the ability to measure acceleration, rate of turn, and determine certain step variables for a prolonged period of time. The patient can wear the pedometer while in his/her normal environment, performs daily routines and ambulates in his/her normal environment, all while the pedometer makes measurements of acceleration, rate of turn, etc. and records the information on the storage unit of the pedometer. In one embodiment, the pedometer can make determinations whether the patient is at risk of falling and alert the patient. In other embodiments, the pedometer, or at least, the storage unit, may be returned to a user clinician and has the information analyzed. The ability to record a patient's gait while in the environment he or she spends a majority of the time will provide more accurate information on whether the patient's gait is abnormal.

A fall is any loss of control during ambulation, i.e., walking, and includes, but is not limited to, stumbling, tripping, loss of balance, loss of muscle control, loss of nerve function, loss of coordination, weakened muscles, and the like.

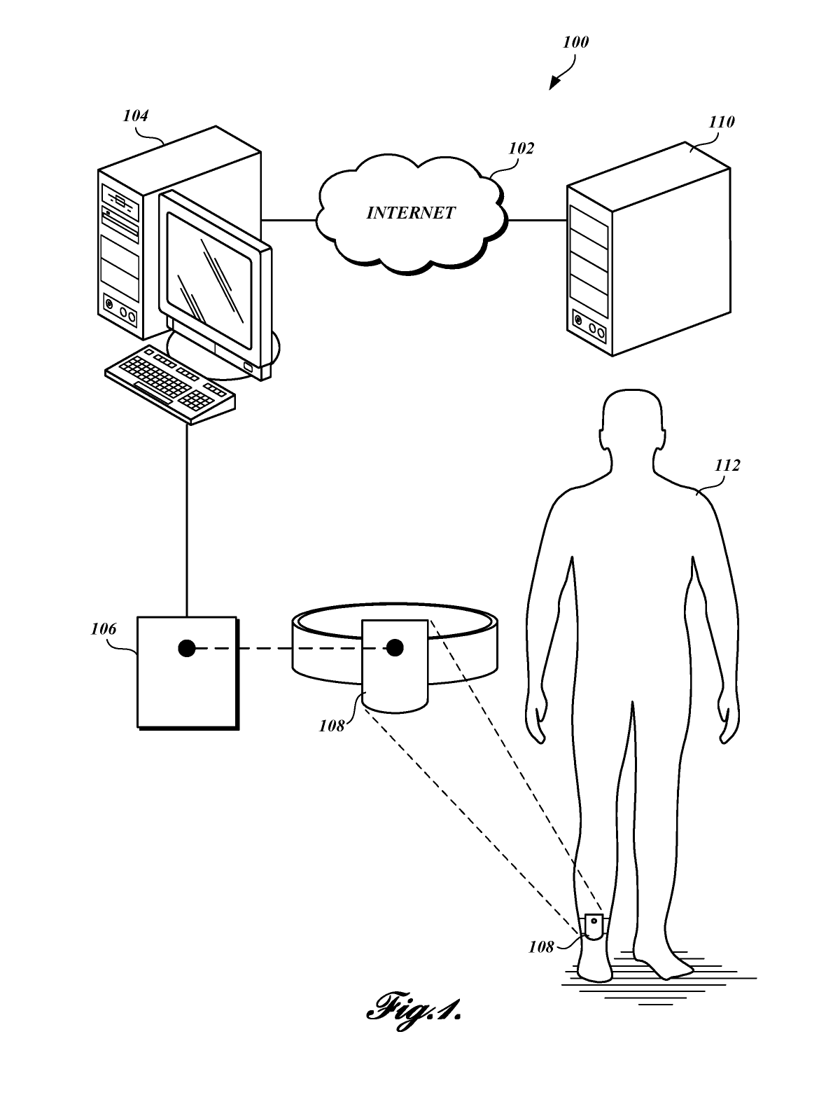

Referring to FIG. 1, one embodiment of a system is illustrated for assessing the fall risk of a person. The system includes a user computer 104 (computer) connected through a communication network, such as the Internet 102, to a server computer 110 (server). The system further includes a docking station 106 (dock) in communication with the computer 104. The system includes a pedometer 108. The pedometer 108 is worn by a patient 112, such as around the ankle.

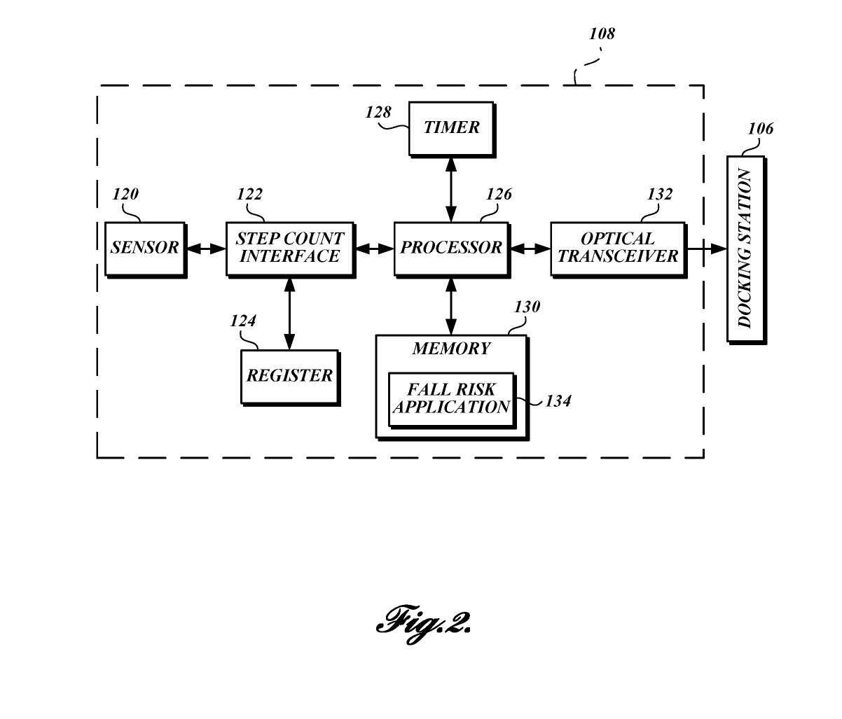

As schematically illustrated in FIG. 2, the pedometer can include, at least, one or more sensors 120, a step count interface 122, one or more registers 124, a processor 126, one or more timers 128, one or more memories 130, and, optionally, an optical transceiver 132. A pedometer having some, but, not all the functionality of the pedometer 108 is described in U.S. Pat. No. 5,485,402, incorporated herein expressly by reference. The differences in structure, operation, and functionality will become apparent from reading the following disclosure.

The pedometer 108 can detect and record the steps the wearer takes over a period of time, and provide the number of steps taken over an interval. During normal walking, a stride includes a stance phase, including the period a foot is in contact with the ground and a swing phase when the foot is not in contact. As explained further below, the pedometer 108 is able to discern a complete stride and also determine the stance and swing phases composing each stride. Furthermore, the pedometer 108 includes sensors, such as accelerometers, or rate gyros, to measure accelerations, rate of turn, and from this information a processor is configured to determine step characteristics of the patient's stride. The pedometer 108 may record the raw information or processed information on a storage unit for downloading to other computers at a later time. The pedometer 108 may include an optical transmitter/receiver to permit the pedometer 108 to be optically coupled to the docking station 106, which, in turn, is connected to the computer 104, thereby allowing transmitting information to and receiving information from the computer 104.

The pedometer 108 may include one or more sensors 120 for sensing the motion of a user, such as acceleration or rate of turn of a patient's body or any part of the body to which the pedometer 108 is attached. The one or more sensors 120 may include a rate gyroscope and/or orientation sensors. A rate gyroscope provides the rate of change of angle with time to measure patient turns. The pedometer 108 may also include a single-axis rate gyro. A single-axis rate gyro will sense the angular rate of change of the ankle in the axial direction (normal to the floor), which is an accurate indication of turning events. It is believed that a greater risk of fall is incurred during turning rather than straight walking. This greater fall risk is accompanied by a greater risk of injury as it introduces much greater likelihood of hip fracture. A suitable accelerometer can be a triaxial accelerometer, such as an ADXL 335 triaxial accelerometer that samples at 300 hertz. However, 200 hertz is also suitable. The sensor may also be constructed from a dielectric angle sensor, or a memory switch. Furthermore, the pedometer 108 may comprise multiple sensors for sensing movement relative to one another. The one or more sensors of pedometer 108 provide information to a step determination unit. The step determination unit is generally software and hardware responsive to the acceleration or other signal for determining whether the wearer has taken a step. The step determination unit includes a step count interface 122 coupled to one or more registers 124. The registers 124 are provided for recording step determination data such as, for example, a minimum acceleration data unit indicating a minimum acceleration required before the activity will be counted as a step, a maximum acceleration data unit indicating a maximum acceleration that will be tolerated before the acceleration signal is discounted, and a minimum time unit indicating the minimum duration that the pedometer 108 must be accelerating before a step will be counted. The pedometer 108 provides the wearer with the ability to program the registers so that the sensitivity of the registers may be more or less in order to increase the accuracy and avoid false positives (step counted when no step taken) and/or false negatives (step taken but not detected). The pedometer 108 includes a storage unit, such as memory 130, for storing the step determination data. The storage unit or memory can be any tangible computer-readable medium. The pedometer 108 includes a timer unit 128, or clock, for determining the time period over which the steps are counted. The memory 130 includes read-only memory (ROM) for storing program and instruction data for controlling the operation of the data processor computer within the pedometer 108. The pedometer 108 also includes random access memory (RAM) for storing data for programming the data processor as well as for recording data provided by the data processor computer. The pedometer 108 may optionally include a fall risk application 134 that compares step variables to model step variables to determine whether the patient is in imminent risk of falling. The memory is also constructed for storing a step rate data unit that indicates the amount of time that the step signal will be ignored after a step is counted. The step rate data unit thereby permits a user to determine a gait, or a step rate (e.g., steps per minute, steps per hour, and the like). To determine the step count data, the data processor counts the number of steps taken during each step rate time interval and records the number into memory. A new step count data unit is provided for each measurement time interval. The measurement time intervals can be consecutive. However, the pedometer 108 may be programmable to specify nonconsecutive time intervals. The length of the measurement time interval may be selected. Additionally, the pedometer 108 can be programmed to begin monitoring at a specific time and end monitoring at a specific time. Alternatively, the pedometer 108 may be programmed to monitor a selected time period of each day for a selected number of days. While the pedometer 108 is active, the pedometer 108 may be sensing and recording acceleration information or rate gyro information, along with times, and storing the information for later retrieval or for use in real-time. The pedometer 108 includes a communication interface, such as an optical transmitter/receiver for transmitting and receiving optical signals, circuits for converting the optical signals to electrical signals and for converting the electrical signals to optical signals. However, the pedometer 108 may employ other means of communicating information to and receiving information from the computer 104. For example, the pedometer 108 may have a wired interface, such as a Universal Serial Bus (USB), or a wireless radio frequency interface, such as Bluetooth. Finally, the pedometer 108 is used to collect step information for use in calculating the fall risk as described further below. Step information is collected from the one or more sensors. Such information may include, but is not limited to, acceleration information, rate gyro information, timing information, and duration information.

The pedometer 108 includes one or more processors 126 for determining the step activity of the wearer and for storing step count data and step information in the storage unit memory 130. The processor 126 may comprise any electronic circuit or circuits for performing the functions discussed herein. The processor 126 is also coupled to one or more timers 128. The timers 128 include a first timer for counting consecutive measurement time intervals wherein each measurement time interval is equal to the time specified by the time interval data unit stored in the memory 130. The timers 128 further include a second timer for counting a step rate time interval wherein each step rate time interval is equal to the time specified by the step rate data unit stored in the memory 130. The timers 128 may further include one or more timers to keep track of the duration of step events, such as heel contact, toe off, stride duration, swing phase duration, stance phase duration, step duration, and the like. Although the timers 128 are shown as a discrete block, those skilled in the art will appreciate that the timers 128 may actually be a portion of the processor 126.

To determine the step count data, the processor 126 counts the number of steps taken during each step rate time interval and records this number into the memory 130. During each step that is counted, the step information from the one or more sensors may be sensed and/or recorded by the pedometer 108. The step information may be used immediately to calculate a risk of falling, or may be retrieved later for analysis. A new step count data unit is provided for each measurement time interval. In one embodiment, the measurement time intervals are consecutive. However, due to the programmable nature of the pedometer 108, many alternatives for specifying the measurement time interval may be provided by the patient. As examples, the length of the measurement time interval may be selected. Also, the pedometer 108 can be programmed to begin monitoring at a certain time and quit monitoring at a selected time. As another alternative, the pedometer 108 may be programmed to monitor a particular time period each day, e.g., 9 a.m. to 11 p.m. daily. The duration of the step rate time interval is programmable as well as the step determination data. Furthermore, the processor 126 may be constructed for compressing the step count data before storing it in the storage unit memory 130.

The pedometer 108 further includes optical transceiver 132. The optical transceiver 132 is provided for transmitting and receiving optical signals, for converting optical signals to electrical signals, and for converting electrical signals to optical signals. The optical transceiver 132 is constructed for coupling the electrical signals to a data bus 308 for communication with the processor 126. The optical transmitter/receiver 132 may be selected from a variety of optical transducers presently available. Further, other types of transducers may be provided to couple the pedometer 108 to the computer 104. As examples, transducers for infrared, sound, electric field, or magnetic field coupling may be provided. Still further, as mentioned above, the housing of pedometer 108 may be constructed to be opened to permit coupling by electric connectors.

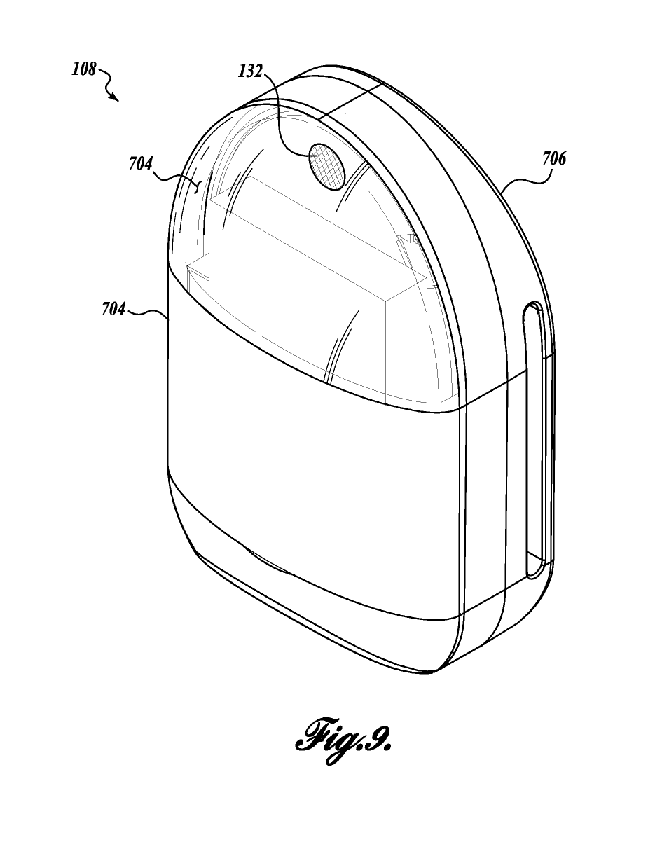

As best illustrated in FIG. 9, at least a portion 700 of the pedometer 108 is constructed of a transparent material. As further illustrated in FIG. 2, the pedometer 108 is constructed to be optically coupled to docking station 106, via a transparent material. The docking station 106 is constructed to be optically coupled to the pedometer 108 to permit a user to program the pedometer 108. Furthermore, the optical coupling between the pedometer 108 and the docking station 106 permits a user to interrogate the pedometer 108 thereby to obtain the recorded information. In one embodiment, the pedometer 108 is provided with programming for analyzing and reporting the step information and assessment of fall risk. In another embodiment, the computer 104 is provided with programming for analyzing and reporting the step information and assessment of fall risk. In another embodiment, the server 110 is provided with programming for analyzing and reporting the step information and assessment of fall risk. In another embodiment, the computer 104 and the server 110 are linked through a network, such that the server 110 stores patient information and performs fall risk assessment, and access to this information is provided to the user computer 104 based on a user fee.

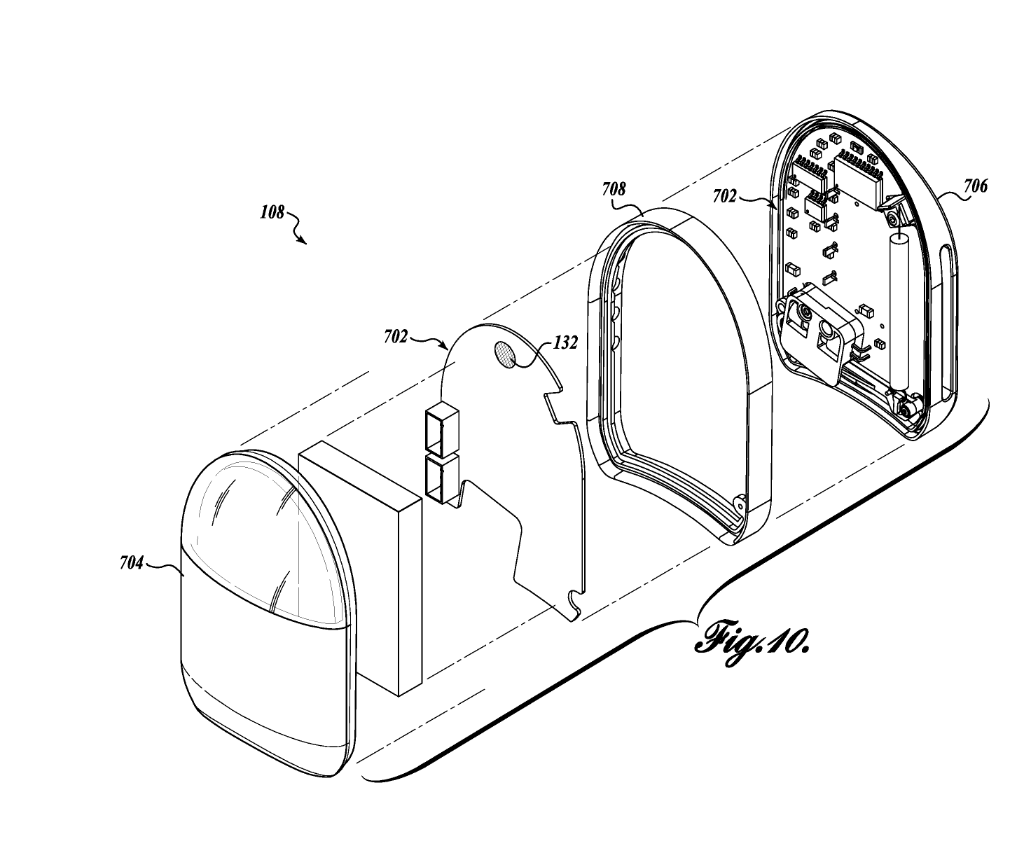

Referring to FIG. 9, a diagrammatical illustration of the pedometer 108 is shown. The pedometer includes the transparent housing portion 700 described above. Referring to FIG. 10, a diagrammatical illustration of the interior of the pedometer 108 is shown. The pedometer 108 may be made from a first and second housing halves, 704 and 706, sandwiching a middle housing 708. The interior includes the components described in relation to FIG. 2. The electronic components may be mounted on one or more boards 702.

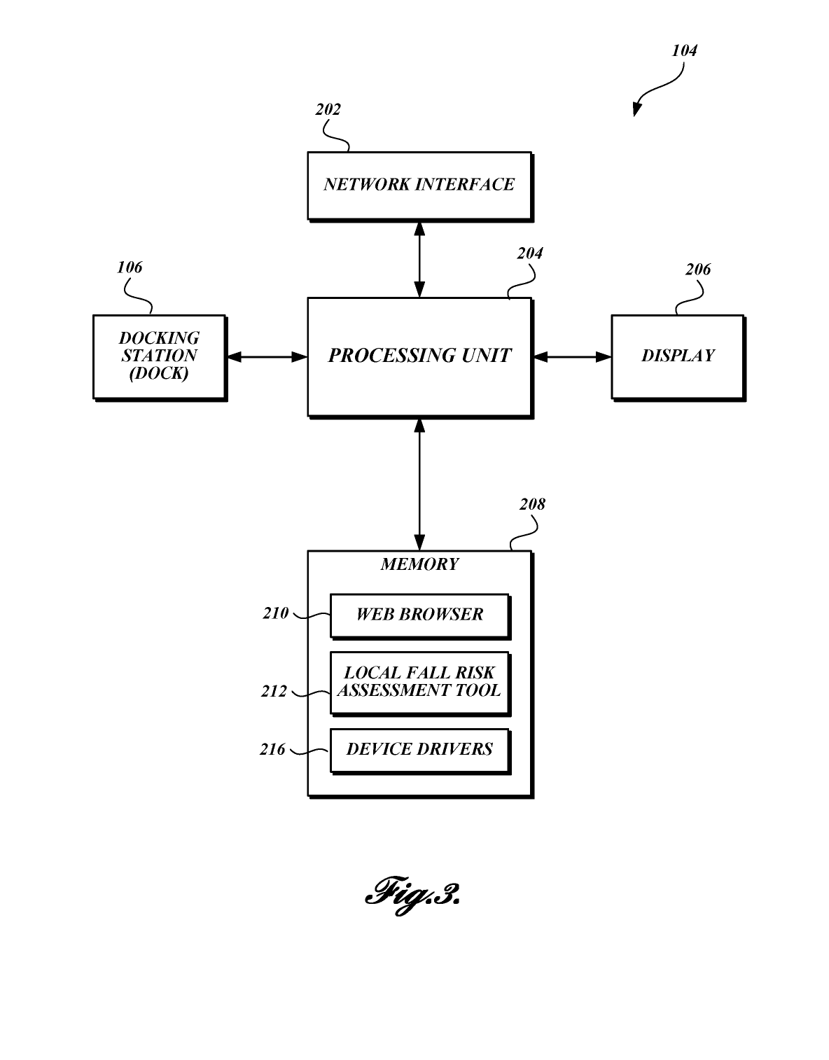

Referring to FIG. 3, the computer 104 includes a processing unit 204, a display 206, a memory 208, and a network interface 202. While shown as a single stand alone computer, persons of skill in the art will appreciate that functions of computer 104 may be distributed among more than one computer, either distributed locally or remotely to one another, and all communicatively linked to each other via a network, such as the Internet.

The computer 104 communicates with the pedometer 108 via the docking station 106. The docking station 106 is constructed from an optical transmitter/receiver that is coupled to an optical interface. The optical interface is constructed to operate in a manner similar to the optical interface 132, with the exception that the coupling between the optical interface and the computer 104 may be through a data port instead of directly to a data bus.

The computer 104 may comprise a standard personal computer having a processing unit 204, display 206, and a means of interfacing, such as a keyboard, mouse, touch screen, light pen, and the like.

The memory 208 generally comprises a random access memory (RAM), a read-only memory (ROM), and a permanent mass storage device, such as a disk drive. The memory 208 stores program code and data necessary for operating a Web browser 210, for running and operating a “local” fall risk assessment tool 212, and various device drivers 216, such as for communicating with the docking station 106. The tools and applications running on the computer may be described in the context of computer-executable instructions, such as program modules being executed by the computer 104. Generally described, program modules include routines, programs, applications, objects, components, data structures, and the like that perform tasks or implement particular abstract data types. “Local” as used herein refers to the computer 104, as opposed to “remote,” which describes the server 110. The Web browser 210 can be any Web browser known in the art such as Netscape Navigator® or Microsoft Internet Explorer®. It will be appreciated that the components in the memory 208 may be stored on a tangible computer-readable tangible medium and loaded into the memory 208 of the computer 104 using a drive mechanism associated with a computer-readable tangible medium, such as a floppy or DVD/CD-ROM drive.

The computer 104 is connected to the server computer 110 through a network, such as the Internet 102. As is well understood, the Internet 102 is a collection of local area networks (LANs), wide area networks (WANs), remote computers, and routers that use the transmission control protocol/Internet protocol (TCP/IP) to communicate with each other. The World Wide Web (www) is a collection of interconnected, electronically stored information located on servers connected throughout the Internet 102. In accordance with one embodiment disclosed herein, a user/clinician using the computer 104 can assess the fall risk of a patient over the Internet 102 via a Web browser by communication to the remote server computer 110 and may pay for receiving a determination and reports relating to a client's assessment of fall risk. The computer 104 can be any number of computer systems, including, but not limited to, work stations, personal computers, laptop computers, personal data assistants, servers, remote computers, etc., that are equipped with the necessary interface hardware connected temporarily or permanently to the Internet 102. Those of ordinary skill in the art will appreciate that the computer 104 could be any computer used by a user/clinician to communicate with the remote server 110 to send and receive information relating to a patient's fall risk. Additionally, those of ordinary skill in the art will appreciate that the computer 104 may include many more components than those shown in FIG. 2. However, it is not necessary that all of these generally conventional components be shown in order to disclose an illustrative embodiment for practicing the present invention. For example, the computer 104 may include an operating system, such as the Windows® operating system. As shown in FIG. 3, the computer 104 includes a network interface 202 for connecting to a LAN or WAN, or for connecting remotely to a LAN or WAN. Those of ordinary skill in the art will appreciate that the network interface 202 includes necessary circuitry for such a connection and is also constructed for use with the TCP/IP protocol, the particular network configuration of the LAN or WAN it is connecting to, and a particular type of coupling medium. The computer 104 is also connected to the docking station 218 via any communication protocol compatible with both the computer 104 and the docking station 218.

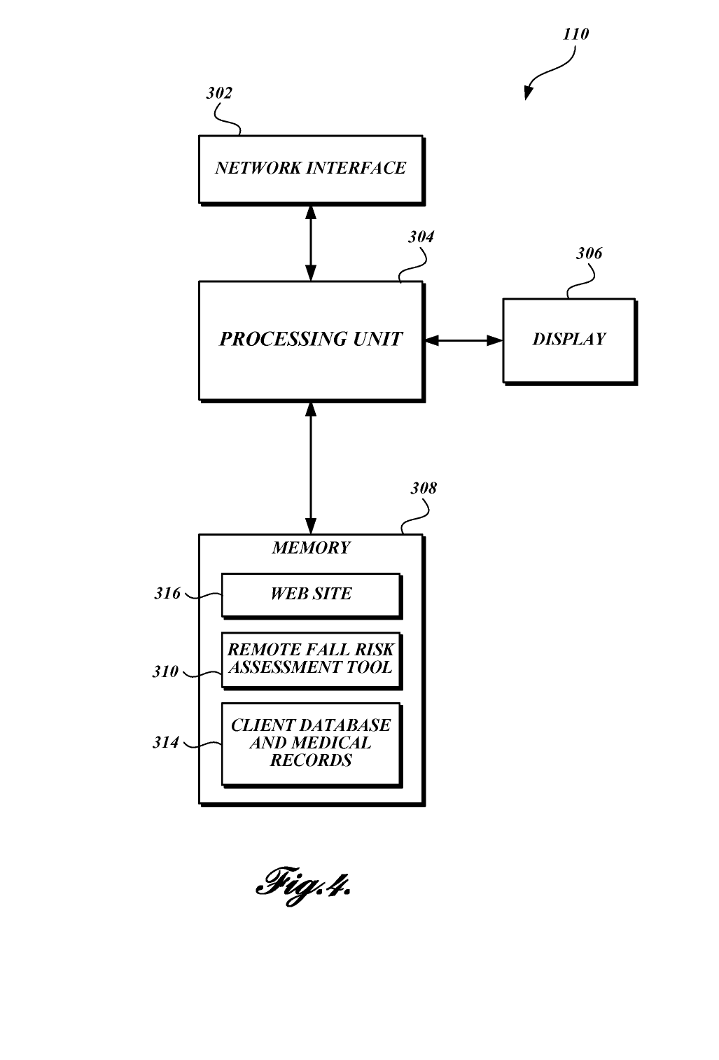

FIG. 4 shows the various components of the server computer 110. Those of ordinary skill in the art will appreciate that the server 110 includes many more components than those shown in FIG. 4. However, it is not necessary that all of these generally conventional components be shown in order to disclose an illustrative embodiment of practicing the present invention. As shown in FIG. 4, the server 110 includes a network interface 302 for connecting to a LAN or WAN, or for connecting remotely to a LAN or WAN. Those of ordinary skill in the art will appreciate that the network interface 302 includes necessary circuitry for such a connection and is also constructed for use with the TCP/IP protocol, the particular network configuration of the LAN or WAN it is connecting to, and a particular type of coupling medium. The server 110 includes a processing unit 304, a display 306, and a memory 308. The memory 308 generally comprises a random access memory (RAM), read-only memory (ROM), and a permanent mass storage device, such as a hard disk drive, tape drive, optical drive, floppy disk drive, or combination thereof. In one embodiment, the memory contains a client and medical records database 314, which includes information relating a list of patients and each patient's medical records, including, but not limited to, step data and stability data and other information and associated reports. The server 110 memory may host a Web site containing a multiplicity of Web pages. The Web site provides a Web service to allow users to manage the medical records of patients, and specifically to determine the fall risk of patients. The memory 308 also contains a remote fall assessment tool 310. “Remote” as used herein is used to denote components found on the server 110, and “local” is used to denote components found on the computer 104. The remote fall assessment tool 310 receives input step data and processes the data and outputs a fall risk level of a patient.

Communications between the computer 104 and the server computer 110 may be encrypted via the generation of an encryption key pair comprising a secret key and a public key. For example, a secure socket layer (SSL) protocol is used for establishing a secure connection. SSL uses public key encryption incorporated into the Web browser 210 and server 110 to secure the information being transferred over the Internet 102. The encryption, decryption, and transmission of encrypted data over the Internet 102 using a public and private key is a well know operation.

Having described the components of a system used to assess the risk of falling, a method to assess the fall risk will be described.

The disclosed method uses the system illustrated and described in FIG. 1. The system uses a pedometer 108, as described in association with FIGS. 1 and 2. The system may include the docking station 106 that can optically receive the data collected by the pedometer 108 and communicate the data to the computer 104. However, in other embodiments, the pedometer 108 may communicate directly with the computer 104 or even the server 110, such as through the Internet. The computer 104 communicates via the Internet 102 with the server 110 to provide the data collected with the pedometer 108 and receives results from the server 110 using the local and remote fall risk assessment tools 212 and 312 stored in the computer 104 and the server 110, respectively. The server 110 provides a service in the form of hosting a Web site to store the list of clients and the clients' medical records, including the data collected using the pedometer 108, provide for the assessment of the fall risk, generate reports, provide for the creation of accounts, provide for the downloading of the local fall risk assessment tool, and collect payment for the use of the service. The local fall risk assessment tool 212 performs such activities as device setup and data reading in connection with the pedometer 108. The remote fall risk assessment tool 310 performs functions such as online remote storage of step data, medical data, and processing the step data, and presenting the results through a Web site for consumption and analysis. The remote fall risk assessment tool 310 also offers the ability to manage client information. Most of the functionality resides on the Web site and can be accessed through the Web browser 210. This allows the local fall risk assessment tool 212 to remain small and easy to install and be used on most of the commonly used computer platforms. All of the communications between the local fall risk assessment tool 212 and the server 110, as well as between the Web browser 210 and the Web site, are encrypted, thus providing for security. The data is securely stored on the server 110. A user/clinician will only have access to the information that they themselves entered into the system. This is managed by creating accounts for each of the users.

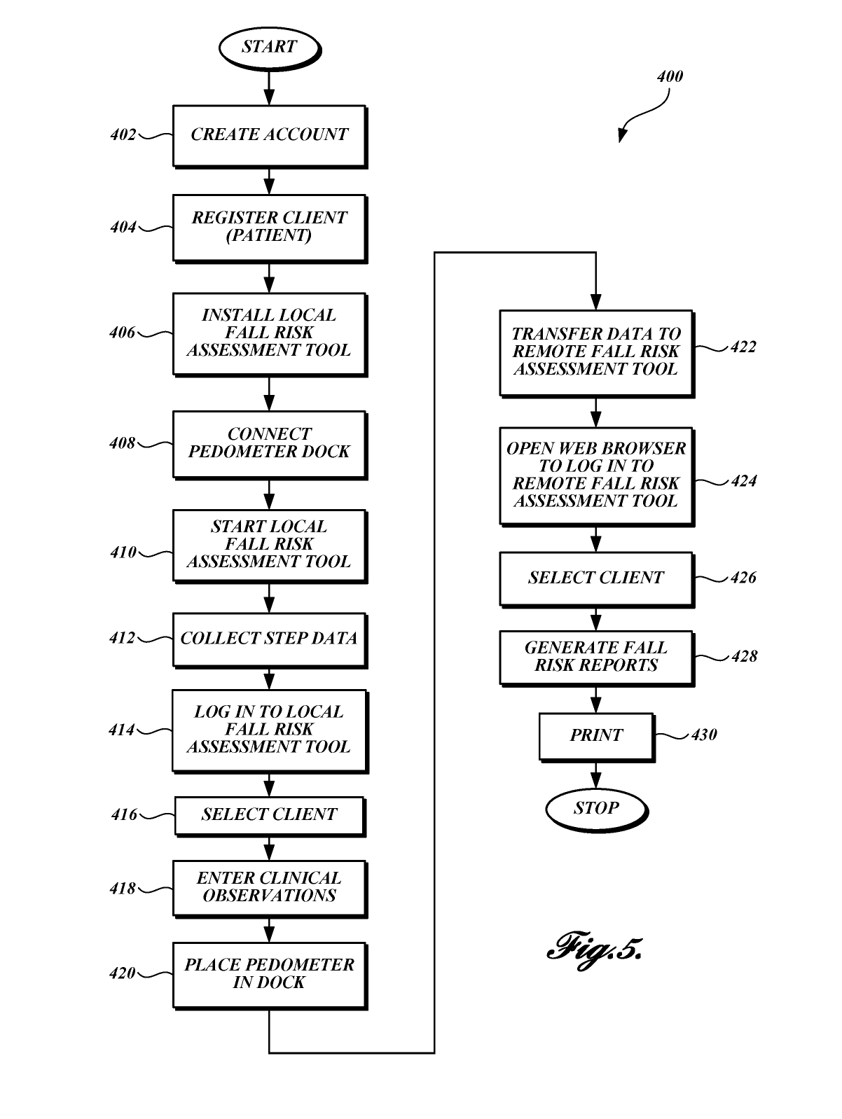

Referring to FIG. 5, a method 400 for assessing the fall risk of a patient is illustrated. Assessing the fall risk is important since knowing the risk of the patient to fall is useful in prescribing the appropriate treatment to minimize the probability. The fall risk assessment as disclosed herein uses the pedometer 108 to gather information relating to step variables.

Step 402 is for creating a user account to use a Web site for determining the fall risk of a patient. The user, a clinician, begins by opening the Web browser 210 on the computer 104 and navigates to a particular Web site that supports a Web service for assessing the fall risk of patients. The server 110 may host the Web site. A Web page may include a menu item entitled “Create Account.” Some of the information may be optional and can be edited at a later point in time. After entering the required and/or optional information, the user account may be created. The Web page may require a user to select a user name and password. In one embodiment, once the user name is chosen, the user name cannot be changed later. Preferably, a strong password is chosen that is case sensitive and contains a minimum of seven characters and at least one non-alphanumeric character. The user is prompted to enter an e-mail address that is unique to the Web site. The Web site checks and verifies that the e-mail is unique. After completing registration, the user will be presented with a successful account creation notice, such as an e-mail confirming the account creation, may be sent to the e-mail address.

Referring to FIG. 4, from block 402, the method enters block 404. Block 404 is for the user to enter client information. Using the Web browser, the user can navigate to a Web page that allows the user to enter information corresponding to each client for which they plan to enter step or moment data. The user may enter the personal information of the client in each field. After the information is added to the data input fields, the patient will be added to the online database 314 in server 110. The Web page allows for clearing all the information at once. The Web page also allows for sorting clients by ID number, first name, last name, diagnosis, and creation date.

Data entered up to this point in the method relates to the creation of a user account and to the creation of a list of an online patient database. In order to begin collecting the step information that will be used to calculate the fall risk, the user is required to load the local fall risk assessment tool onto the user computer 104. It is common practice to download applications by establishing a connection to the Internet 104 and then downloading the application onto the user computer 104. From step 404, the method enters step 406. In step 406, the user can download and install the local fall risk assessment tool from the Web site 316 and configure the computer 104 to operate the docking station 106. Part of the installation may include installing device drivers needed to communicate with the docking station 106 and a serial port driver, such as USB. The docking station 106 may be physically connected to the computer 104 through a USB cable. The computer 104 has an operating system such as the Windows® operating system. The operating system may automatically detect the connection to a new device and search for the appropriate device driver. From step 406, the method enters step 408, for connecting the pedometer dock 106.

After the hardware and software are installed and configured, the user may then start the local fall risk assessment tool in step 410. As discussed above, preferably the pedometer 108 is programmable to receive instructions concerning the duration and intervals over which steps, step acceleration, and rate of turn information is to be recorded, including the start and the stop times. As part of the pedometer setup, the user/clinician may enter physical parameters of the patient, such as age, sex, walking speed, height, weight. The pedometer 108 may consider these parameters in its algorithm to decide whether or not a step has, in fact, been completed, or in comparing the step variables of the patient to the model step variables. The pedometer 108, and one or more of the computers may also use patient parameters in the assessment of fall risk. For example, the actual step variables that are calculated for the patient are compared to model step variables that are compiled from step information from persons whose parameters match those of the patient. For example, in one embodiment, the patient step variables are compared to model step variables that are compiled from persons matching anyone or more of the patient's age, height, weight, sex, and walking speed. In other embodiment, the patient's step variables can be normalized and compared to normalized model step variables. In another embodiment, the patient's step variables from one information-gathering session can be compared to the same patient's variables from a later information-gathering session. This allows the user/clinician to notice any loss or deterioration of functionality in the patient's gait or stride. In step 410, the user starts the local fall risk assessment tool 212 to begin the process of recording of step data.

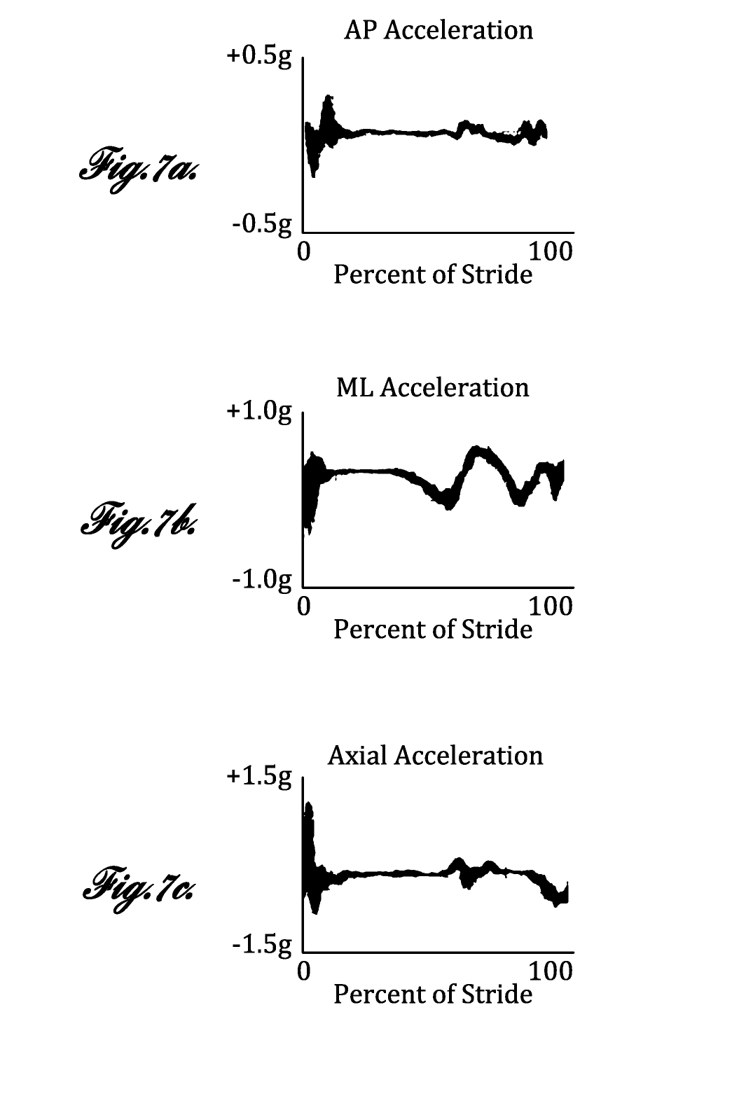

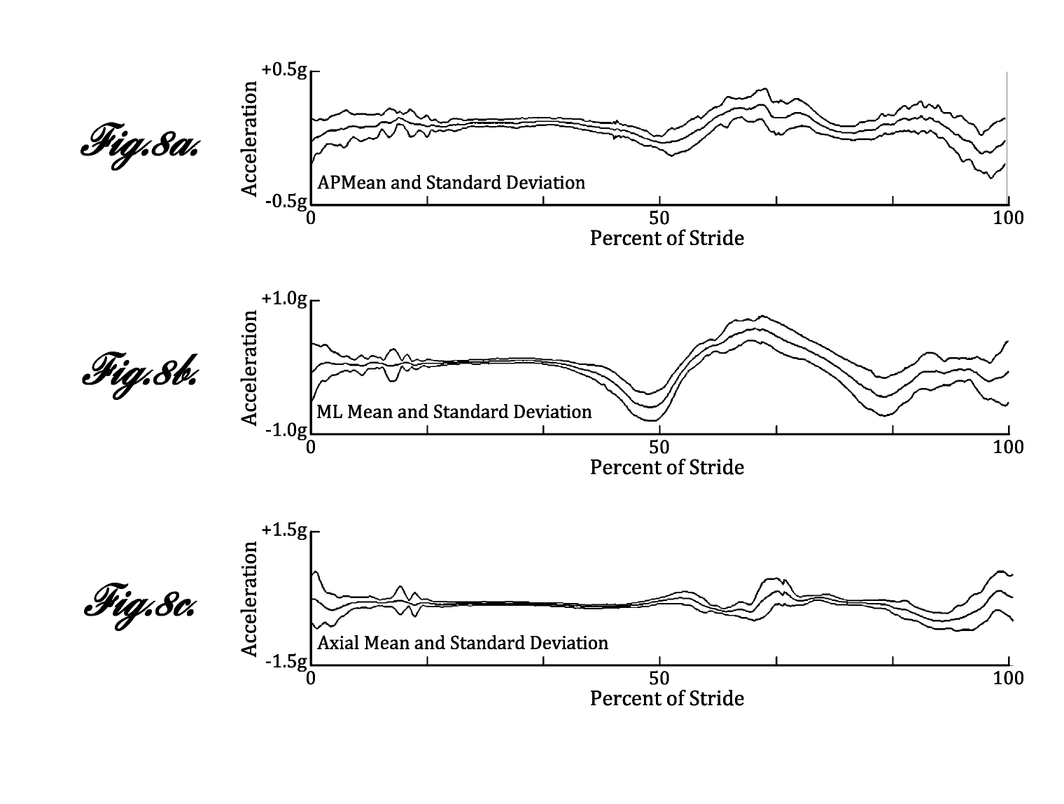

From step 410, the method enters step 412. In step 412, the patient is asked to wear the pedometer 108, such as on the ankle. During step 412, the patient collects the step information. The pedometer 108 may be worn by the patient continuously, day and night, for the selected period of time. During data collection, the patient is in his or her normal environment, such as the environment that the patient occupies for the majority of the day. This may include the home, neighborhood, commonly visited places of commerce, and the like. Collecting data from the patient while the patient is in his or her normal environment is preferable to collecting step data while in a clinical setting. The pedometer 108 first checks whether it is time to look for steps. If it is determined that the pedometer 108 is ready to check whether steps are occurring, thereafter, every time the patient completes a step, the pedometer 108 will count the step and may note the time interval in which it was recorded. Additionally, the time may also be recorded. Throughout each step, the pedometer 108 may also be measuring the accelerations in three orthogonal axes, the rate of turn, and other information depending on the sensors, among with time and duration. The accelerations, the rate of turn, and other information is also time stamped, so that accelerations may be recorded versus time. After the recording period is at an end, the patient may return the pedometer 108 to the user/clinician. FIG. 7A is a representative graph of anterior/posterior acceleration for a stride. FIG. 7B is a representative graph of medial/lateral acceleration for a stride. FIG. 7C is a representative graph of axial acceleration for a stride. FIG. 8A is a representative graph of the mean (center line) of anterior/posterior acceleration for a stride with the standard deviation upper and lower spread. FIG. 8B is a representative graph of the mean (center line) of medial/lateral acceleration for a stride with the standard deviation upper and lower spread. FIG. 8C is a representative graph of the mean (center line) of axial acceleration for a stride with the standard deviation upper and lower spread.

From step 412, the method enters step 414. Step 414 is for logging into the system to begin downloading the data to the online database 314. Once the patient has worn the pedometer 108 for the selected period of time and has returned the pedometer, the data may be downloaded from the pedometer 108 and uploaded to the Web site 316. This process is carried out using the computer 104 connected to the Internet 102 and the local fall risk assessment tool 212. The pedometer 108 may be placed alongside the dock 106 to enable optical communications from the pedometer 108 to the dock 106. The user may once again start the local fall risk assessment tool 212. The local fall risk assessment tool 212 will ask the user to log into the system using the previously created account. The user enters the user name and password for the account. After successfully logging in, the list of patients that have been previously registered may be displayed on the computer display 206. In step 416, the user selects the patient whose data is to be uploaded. If the patient is not in the database, a new patient may be created. The same procedure as described for adding a new patient will start.

Step 418 is for entering clinical observations. Step 418 is optional. In step 418, the local fall risk assessment tool 212 will ask the user to input their assessment of the patient's risk of falling as a scalar value or category, such as low, medium, or high risk.

After entering the patient information, the user will be asked to confirm that the pedometer has been placed on the docking station 106. Step 420 is for placing the pedometer 108 alongside the dock 106. The fall risk assessment tool 212 may prompt the user to verify the correct placement of the pedometer 108. Once the user confirms the pedometer 108 is correctly placed on the dock 106, step 422 is entered for reading the data and transmitting the data over the Internet 102 to the remote server 110. When the data upload is complete, the user may be notified the data transfer has been successfully completed by displaying a notification window.

From step 422, the method enters step 424. Step 424 is for opening the Web browser to log onto the Web site 316 associated with the remote fall risk assessment tool 312. The local fall risk assessment tool 212 may be used to open the Web browser to communicate to the server 110. The user navigates via a Web browser to log into the Web site 316 to gain access to the remote fall risk assessment tool. The user logs into the Web site 316 using the same user name and password as the local login. A Web page may be displayed to the user on the computer 104. To receive an assessment of the fall risk, the user can select the name of the patient about whom the user desires to receive a report. The patient may have a plurality of data sets that have been uploaded for various recording sessions. The user will be able to distinguish among the data sets based on the recording interval or dates. The user has the option of selecting which data set to be analyzed for fall risk from the list of reports. Additionally, the user may compare one data set to another data set. For example, the user is able to compare an earlier data set to a later data set. This allows the user to notice signs of improvement or to notice loss or deterioration of functionality over time.

The first time a particular report is requested, the user may have to pay a user fee to receive the report. Transactions involving payment in exchange for goods over the Internet has become a common channel for providing goods to users of such goods. The Web site 316 disclosed herein uses any of the secure forms of payment for such transactions. Following the initial payment for a report for one data set, for example, the user will be able to access the report at any time in the future for no additional charge. The user may print and save the report.

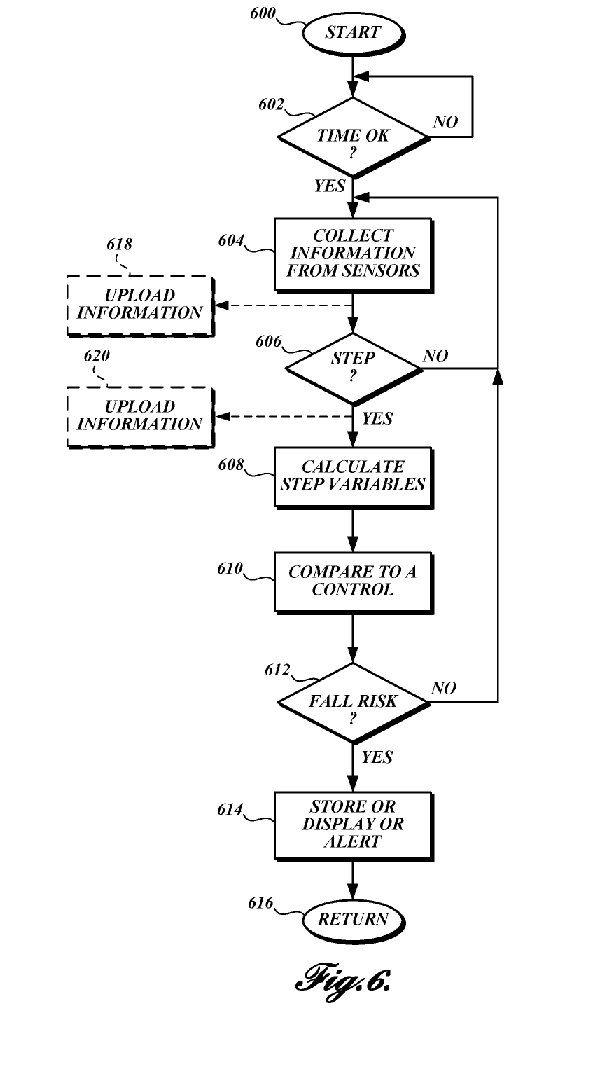

Referring to FIG. 6, a method for assessing the fall risk is illustrated. The method illustrated in FIG. 6, may be performed by one or more computers, such as computer 104 or 110. Alternatively, the method of FIG. 6 may be performed by the pedometer 108 by the fall risk application 134. Alternatively, the method may be performed by one or more computers and the pedometer 108. The method may be iterative, or the method may perform one or more blocks, repeat one or more blocks, and then continue with the remaining blocks. In some embodiments, not all the blocks are performed or the blocks may be performed in a different order. In one embodiment, once the time is validated for the start of collecting step information, the method is performed each time and/or at the conclusion of each step or stride. In this manner, the method can determine in real time whether the last stride or step indicates a fall risk and alert the patient of the risk immediately. The method can be performed by the pedometer each time the patient takes a step/stride. Alternatively, the pedometer can be used mainly for information gathering, and the assessment of fall risk is done by one or more computers after the pedometer is returned from the patient to the user/clinician.

The method starts in block 600. From block 600, the method enters block 602. In block 602, the pedometer 108 determines whether the time to start searching for step information is appropriate. As described above, the pedometer 108 can be programmed to record during certain periods of the day. From block 602, the method enters block 604, the pedometer 108 is active and starts to collect information from the one or more sensors. The pedometer 108 is able to determine when a step begins at the moment of heel contact with the ground and ends with the toe off the ground, based on the accelerometers, for example. The period of contact between heel contact to toe off is defined as the stance phase, and the period between toe off and the next heel contact is defined as the swing phase. The stance phase and swing phase define a step, and the right foot step and left foot step define a stride, i.e., two steps. Step 606 is meant to represent one or more than one step. When the time is okay for collecting data, the pedometer is continuously monitoring the sensors for step activity. When a step occurs, the pedometer 108 recognizes a step and stores the information relating to the step. If a step did not occur, the pedometer may discard or store the information, such that it can be distinguishable from the “true” step information. The pedometer 108 is able to delineate each step from heel contact to heel contact, and is able to delineate a stance phase from a swing phase. The pedometer 108 is able to determine the accelerations occurring during each stance phase and each swing phase for each step. The pedometer is able to determine the rate of turn of the patient using the rate gyro. It should be understood that a step may be measured from any point in the stride. In step 606, the pedometer 108 makes a determination whether a step has occurred. If the determination is no, the method returns to block 604 to wait for new data. If the determination is yes, a step has occurred, the method enters block 608.

In step 608, the method calculates step variables. In one embodiment, one or more computers, such as 104 and 110 may determine the step variables for a patient's walking session. This is accomplished after the uploading of stored information from the pedometer 108 to the computers 104 and 110, as indicated by block 618. However, in other embodiments, the fall risk application 134 of the pedometer 108 is able to discern and measure the step variables for each step or for a collection of steps. Block 608 is meant to represent that one or more computers or the pedometer 108 may calculate one or more of the following step/stride variables: mean stride duration, mean step duration, standard deviation of stride duration, standard deviation of step duration, mean swing duration for all steps, standard deviation of swing duration, stride standard deviation of anterior/posterior acceleration, stride standard deviation of medial/lateral acceleration, stride standard deviation of axial acceleration, swing standard deviation of anterior/posterior acceleration, swing standard deviation of medial/lateral acceleration, swing standard deviation of axial acceleration, swing/stance transition standard deviation of anterior/posterior acceleration, swing/stance transition standard deviation of medial/lateral acceleration, and swing/stance transition standard deviation of axial acceleration. Variability of each variable from stride to stride or step to step may also be calculated. A measure of variability may include duration, i.e., the difference in time it takes to complete one stride (or step or a phase of a step, such as swing or stance phase) compared to other strides (or steps or phases of a step). A measure of variability may include acceleration, i.e., the difference in acceleration between one stride (or step or phases of a step, such as swing or stance) compared to other strides (or steps or phases of a step) along one to three axes. These variables may be calculated as normalized standard deviations (coefficient of variation). Variables with statistical differences using an unpaired T-test (p≦0.05) can be used in a receiver operator characteristic curves to determine the best combination of measurements that result in the most ideal sensitivity and specificity for differentiation of the fall risk group. In one embodiment, the coefficient of variability of swing phase is a measure able to distinguish the fall risk group from the non-fall, i.e., the low risk group with a p=0.03 (a statistical probability that there is only a 3% chance that the measured effect was not real, i.e., by chance). Without intending to be bound by theory, this may mean that non-fallers are better able to adapt to perturbations in the community than people at risk of falling, while during continuous controlled walking, lower swing duration variability may indicate more stability because less stride correlation is needed. A cutoff value of 17 for a receiver operator characteristic (ROC) analysis using swing duration coefficient of variation would result in 100% sensitivity (100% detection of fall risk persons) and 75% specificity (25% chance of misclassifying a non-fall, low risk subject as a fall risk). ROC is a standard measure for ability to distinguish a condition from data. The cutoff values are provided as guidelines, and are generally accepted as adequate in medicine. Swing variability is at least one variable that can be used as a parameter in assessing whether a patient is at risk of falling. However, any one or a combination of variables or the variation between variables may be used. After computing one or more step/stride variables, the method enters block 610. Block 610 can be performed by one or more computers, such as 104, 110, after uploading of information as indicated by block 620, or by the pedometer 108

An analytical approach to assessing fall risk is based by comparing one or more step variable from a patient to a model step variable. In one embodiment, the model step variable is compiled from one or more persons who are considered to be at little to no risk of falling. In another embodiment, the step variable used in the comparison may be the patient's own step variable from an earlier (or later) session. In one embodiment, the model step variable is compiled from a data set of a number of persons, all considered to be good samples of persons at low risk of falling. Thus, the one or more step variables from a patient being assessed for fall risk are compared to one or more step variables from persons considered to be at low risk for falling. Variables that may be used to determine low risk include, but are not limited, to low stride to stride variability of leg acceleration or gait event duration. In one embodiment, training data is collected from an aged-matched group that are not at risk of falling. The training data may be compiled from any number of subjects. The subjects may wear the pedometer 108 to collect data.

In one embodiment, comparisons of stride and swing time variability are assessed using the information collected from a patient to the information compiled from a sample of persons considered to be good samples who are not at risk for falling.

In one embodiment, a statistical analysis comparison tool uses Lyaponov exponents. This method of unfolding time series data examines the rate of divergence of a measure from its previous state-space. Any variable can be used, from stride timing to medial/lateral acceleration, and evaluated for the rate at which it diverges from a given trajectory (neurosensory system error detection) and the rate at which it converges on optimal (musculoskeletal system response).

In one embodiment, a statistical analysis comparison tool uses Floquet numbers. This method uses a non-linear dynamical approach to examine dynamic stability during gait.

In another embodiment, a statistical algorithm including detrended fluctuation analysis is used. This process calculates fractal scaling indexes as a way to quantify correlations in stride fluctuations with time. Fractal scaling index is a stride dynamic measure that discriminates between fallers and non-fallers with higher level gait disturbance while average time, stride variability, and other measures cannot. A fractal scaling index may be a unique measure that may help to identify fall risk subjects.

In another embodiment, fall risk is correlated to stumble detection. Stumble detection is challenging to detect in the short duration clinical test. However, stumble detection is made possible with the pedometer 108 taking measurements in the patient's normal environment. Stumble events tend to be less memorable for patients than falls and are subject to patient judgment and bias as to what constitutes a stumble. Accelerometry may be used to identify stumbles. For indicating a stumble, a vertical maximum peak-to-peak acceleration, mean plus or minus standard deviation, 0.69±0.26 (stumble) and 0.34±0.19 (non-stumble) may be used. Assuming these means and standard deviations, with an assumed Pearson correlation of r=0.60 between the stumble and the non-stumble pairs, or repeated measurements, an n=10 healthy subjects may provide greater than 99% power using a two-sided, α 0.05 comparison. An N=8 elderly subjects may provide 98% power.

A method to detect stumbles may be as follows. First, a sample size of suitable numbers of good subjects will be utilized to in the algorithm development. Since the elderly may have different stumble recovery techniques, a number of subjects from the fall risk group and a number of subjects from a non-fall risk control group can be used. Subjects may walk on a treadmill while using a safety harness to prevent actual falls. The walking speeds may be the subject's self-selected slow, normal, or fast walking speeds. At each pace, a subject will walk for approximately two minutes without obstacles and two minutes with enough obstacles to cause at least three stumbles. The obstacles may be comprised of empty shoe boxes, shoe boxes filled with stones, ropes, and cylindrical carbon or plastic rolls, for example. A sheet may be used to prevent the subject from seeing his or her legs and the potential obstacle placed on the treadmill. An observer can be used to record when the subject has a stumble, which may be defined as a loss of balance that would have resulted in a fall if corrective measures were not taken or the harness would not be there to support them. Kicks, step-overs, and stops may not be counted as stumbles.

The pedometer 108 may have on-board stumble and fall detection algorithms. For each acceleration parameter, the range of threshold values for distinguishing between stumbles and non-stumble segments may be plotted by using a Receiver Operating Characteristic (ROC) curve. Actual stumbles noticed by the observer may be used as the gold standard. The threshold value that results in the best sensitivity and specificity for detecting stumbles can be selected. The algorithm performance can be examined for each parameter separately and for up to three parameter combinations. The parameter combinations may be evaluated when all parameters are above their threshold and when at least one parameter is above its threshold. The threshold values can be internally validated using bootstrap validation to assess the reliability of threshold values. The computation of test characteristics for documenting the algorithm's performance (positive and negative likelihood ratios, ROC area, sensitivity, and specificity) may be done by applying the algorithm to produce a binary predictor (predicted stumble or no stumble), which is then compared to the gold standard (actual stumble or actual no stumble). Robust variance estimates, which account for the correlation induced by using repeated measurements within each subject, may be used to produce 95% confidence intervals for the test characteristics. The estimates may be separately made for young and elderly patients.

The algorithm yielding the best sensitivity and specificity of detection can be incorporated into the on-board fall risk assessment tool of the pedometer, or used by one or more computers.

Fall risk can by a measure of the deviation or variance of the actual data collected from a patient compared to a model. To analyze for fall risk, the analysis may take certain step variables into consideration. One or more of the step variables are then applied to the model of fall risk using a statistical analysis tool.

The equations used in deriving the model fall risk are derived heuristically to minimize an external criterion called the prediction error sum of squares, or PESS, for previously measured gait stability factors of acceleration and timing of gait events.

$$PESS = \frac{1}{N} {\sum }_{t = 1}^{N} {( {y}_{t} - f ( {x}_{t} , {\hat{a}}_{t} ) )}^{2} \qquad ( 1 )$$

Where N is the number of step variable samples available, Y is the target stability or risk of falling, and a is an estimation of the combined parameters that describe the instability or risk of falling. The equation derivations are achieved using the group method of data handling described by Madala and Ivakhnenko (Madala, H., and A. Ivakhnenko, “Inductive Learning Algorithms for Complex Systems Modeling,” CRC Press, Boca Raton, Fla., U.S.A., 1994), fully incorporated herein expressly by reference. Solving the derived model equations with the step variables results in a numeric estimation of the fall risk. For robustness, estimations from each of the equations become a vote added to a more generalized estimation of the stability. Stability or decreased fall risk is signified by decreased variability in step to step movement sessions time plots. A unit-less (nondimensional) index number can be assigned based on population statistics.

While illustrative embodiments have been illustrated and described, it will be appreciated that various changes can be made therein without departing from the spirit and scope of the invention.

1. A method for assessing the risk of a patient to fall, comprising:
attaching a pedometer on a patient, wherein the pedometer includes one or more sensors;
allowing the patient to engage in activities throughout a predetermined period of time in, at least, an environment the patient occupies for a majority of the day while the pedometer senses information relating to steps taken by the patient, wherein the information includes a measure of variability of a trajectory of a leg during the swing phase of gait with an accelerometer;
with one or more computers, calculating at least one step variable from the information;
with one or more computers, comparing the at least one calculated step variable to a model step variable; and
with one or more computers, providing an assessment of the risk of the patient to fall, wherein the risk is a probability and not a detection of an actual stumble or fall; and
prescribing an intervention action to minimize the probability of the patient to fall.

2. The method of claim 1, wherein the model step variable is compiled from information of persons considered to be samples of low risk of falling.

3. The method of claim 1, wherein the information includes information of acceleration along one or more axes of the patient's foot.

4. The method of claim 1, wherein the information includes information of rate of turn of the patient.

5. The method of claim 1, comprising calculating a variability of stride duration of the patient and comparing to a variability of a model stride duration compiled from a group of persons characterized at low risk of falling.

6. The method of claim 1, comprising calculating a variability of stance phase duration of the patient and comparing to a variability of a model stance phase duration compiled from a group of persons characterized at low risk of falling.

7. The method of claim 1, comprising calculating a variability in accelerations in three orthogonal axes of the patient and comparing to a variability of model accelerations in three orthogonal axes compiled from a group of persons characterized at low risk of falling.

8. The method of claim 1, comprising calculating a variability in stride length of the patient and comparing to a variability of a model stride length compiled from a group of persons characterized at low risk of falling.

9. The method of claim 1, comprising calculating a rate of turn variable and comparing to a rate of turn variable compiled from a group of persons characterized at low risk of falling.

10-11. (canceled)

12. The method of claim 1, further comprising transferring recorded information from the pedometer to the one or more computers, and with the one or more computers comparing the at least one step variable to the model step variable.

13. The method of claim 1, wherein two or more computers are connected to a network, and transferring the assessment of the risk of the patient to fall over the network from a first computer to a second computer.

14. A method for alerting a patient of a risk of falling, comprising:
attaching a pedometer on a patient, wherein the pedometer includes one or more sensors and a processor;
allowing the patient to engage in activities in, at least, an environment the patient occupies for a majority of the day while the pedometer senses information relating to steps taken by the patient, wherein the information includes a measure of variability of a trajectory of a leg during the swing phase of gait with an accelerometer;
with the pedometer, calculating at least one step variable from the information;
with the pedometer, comparing the at least one calculated step variable to a model step variable compiled from a group of persons characterized at low risk of falling; and
when a risk of falling is detected, the pedometer alerts the patient, wherein the risk is a probability and not a detection of an actual stumble or fall; and
prescribing an intervention action to minimize the probability of the patient to fall.

15. (canceled)

16. The method of claim 14, wherein the acceleration information includes information of acceleration along one or more orthogonal axes.

17. The method of claim 14, wherein the information includes information of rate of turn of the patient.

18. The method of claim 14, comprising calculating a variability of stride duration of the patient and comparing to a variability of a model stride duration compiled from a group of persons characterized at low risk of falling.

19. The method of claim 14, comprising calculating a variability of stance phase duration of the patient and comparing to a variability of a model stance phase duration compiled from a group of persons characterized at low risk of falling.

20. The method of claim 14, comprising calculating a variability in accelerations in three orthogonal axes of the patient and comparing to a variability of model accelerations in three orthogonal axes compiled from a group of persons characterized at low risk of falling.

21. The method of claim 14, comprising calculating a variability in stride length of the patient and comparing to a variability of a model stride length compiled from a group of persons characterized at low risk of falling.

22. The method of claim 14, comprising calculating a rate of turn variable and comparing to a rate of turn variable compiled from a group of persons characterized at low risk of falling.

23-34. (canceled)

35. A method for assessing the risk of a patient to fall, comprising:
attaching a pedometer on a patient, wherein the pedometer includes one or more sensors;
allowing the patient to engage in activities throughout a predetermined period of time in, at least, an environment the patient occupies for a majority of the day while the pedometer senses information relating to steps taken by the patient;
with one or more computers, calculating at least one step variable from the information;
with one or more computers, comparing the at least one calculated step variable to a model step variable; and
with one or more computers, providing an assessment of the risk of the patient to fall, wherein the risk assessment is calculated using receiver operator characteristic analysis with a cutoff value that results in detecting about 100% of persons at risk of falling and a less than 25% chance of misclassifying a person at low risk of falling.
